(19) United States
(12) Patent Application Publication
(10) Pub. No.: US 2017/0304467 A1
(43) Pub. Date: Oct. 26, 2017

(54) BLUE DYED CHLORHEXIDINE ANTIMICROBIAL COMPOSITION FOR SKIN DISINFECTION

(71) Applicant: NEX MEDICAL ANTISEPTICS S.R.L., Casorezzo (Milano) (IT)

(72) Inventors: Silvio DANELUZZI, Casorezzo (Milano) (IT); Carlo BIGNOZZI, Ferrara (IT)

(21) Appl. No.: 15/518,066

(22) PCT Filed: Oct. 13, 2014
(86) PCT No.: PCT/IT2014/000268

§ 371 Date: Apr. 10, 2017

(51) Int. Cl. A61K 49/00; A61K 31/155; A61K 9/08; A61K 9/00; A01N 47/44; A61K 47/10; A01N 33/12

(52) U.S. Cl. CPC A61K 49/006; A61K 47/10; A61K 31/155; A01N 33/12; A61K 9/0014; A01N 47/44; A61K 9/08

(57) ABSTRACT

The present invention relates in general terms to an antimicrobial composition having chlorhexidine and a dye selected from 3,7-bis(dimethylamino)-phenothiazin-5-ium chloride and a sodium or calcium salt of [4-(α-(4-diethylaminophenyl)-5-hydroxy-2, 4-disulfophenylmethylidene)-2,5-cyclohexadien-1-ylidene] diethylammonium hydroxide usable in the medical field. In particular, the invention relates to the composition in the form of aqueous solution, hydro-alcoholic solution or gel, and its use as a coloring antiseptic and disinfectant agent, for example in pre-surgical procedures.

### DESCRIPTION

The present invention relates, in general terms, to a blue dyed antimicrobial composition comprising chlorhexidine and a dye usable in the medical field. In particular, the invention relates to said composition as aqueous or hydro-alcoholic solution, or even in a gel form, and its use as a disinfectant and/or antiseptic agent, for example in the pre-surgical procedures.

### STATE OF THE ART

Chlorhexidine is a chemical disinfectant endowed with antiseptic action, having the following formula:

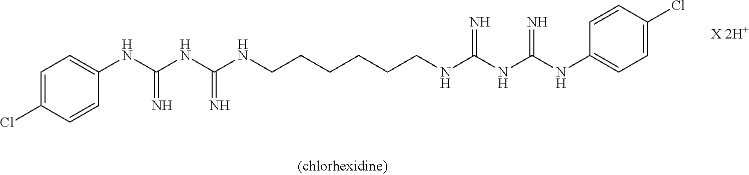 [SMILES: N=C(NCCCCCCNC(=N)NC(=N)Nc1ccc(Cl)cc1)NC(=N)Nc1ccc(Cl)cc1]

Chlorhexidine is characterized by the fact of having a broad spectrum of action, being active towards Gram-positive bacteria and Gram-negative bacteria, as well as to fungi. Chlorhexidine has a bactericidal activity and acts by dramatically increasing the permeability of the bacterial cell membrane, thus altering the protein structure. This causes the precipitation of several cytoplasmic macromolecules, and the subsequent cell death, by lysis of the bacterial cell or the fungus.

The disinfectant solutions on the market are normally chlorhexidine di-gluconate compositions, where the double positive charge, localized on the protonated chlorhexidine, is balanced by the negative charges supported by two anions gluconate: (HO)CH2^{—}[CH₂ (OH)]₄—COO^{—}. This fact is related to the high solubility of the salt which is much greater than the corresponding forms containing anions such as acetate or chloride.

Various combinations of chlorhexidine with other active ingredients are also present on the.market, in order to enhance the antimicrobial action, such as: chlorhexidine+benzalkonium chloride; chlorhexidine+cetrimide; chlorhexidine+didecyldimethylammonium chloride; chlorhexidine+ethanol; chlorhexidine+hydroxyquinoline and chlorhexidine+sodium fluoride.

Chlorhexidine-based preparations are widely used in dentistry, urology, dermatology, gynecology, veterinary medicine and surgery procedures.

Alcoholic solutions of chlorhexidine 2% are, in particular, used in skin antisepsis and in the prevention of surgical site infections.

In preoperative procedures, chlorhexidine is sometimes associated with dyes, these latter mainly used in order to better detect and indicate the disinfected area which will then be subjected to a surgical treatment. In some invasive surgery, such as biopsies and the like, it is, in fact, required the use of coloring solutions or compositions that are able to visually identify the area to treat, also exploiting an antibacterial action as required by the surgery operation itself.

For this purpose, the chlorhexidine can be used in solutions comprising, for instance, the chlorhexidine digluconate salt and a red dye, such as the azorubine (also called E 122) to unequivocally visualize and identify the disinfected area, that will be the object of the surgery.

The use of sulfonated azo dyes such as Azorubine has, however, the disadvantage of causing the precipitation of chlorhexidine in hydro-alcoholic solutions, thus drastically reducing its antimicrobial activity. For this reason, it is often necessary to prepare the coloring antiseptic solution in the time period immediately prior to the use, so as to prevent the rapid precipitation (and thus deactivation) of the components. A similar deactivation also occurs when chlorhexidine is in contact with soaps or toothpaste containing the lauryl sulphate anion, which also causes the precipitation of the chlorhexidine cation.

On the other hand, the use of dyes containing anthocyanins, betalains or porphyrins does not allows to reach a color intensity suitable for an application in the medical field. Also, these dyes do not allow an appreciable marking of the skin, to such an extent to be usable in surgical procedures.

Therefore there is still the need to obtain stable antimicrobial (disinfectants and antiseptics) solutions comprising the chlorhexidine cation and at least one dye having a high color intensity and high stability over the time, to be used, in particular, in the pre-surgical procedures.

The applicants have now surprisingly found that by using a composition comprising chlorhexidine and a dye selected from: chloride, 3,7-bis(Dimethylamino)-phenothiazin-5-ium chloride of formula (I), also known as Methylene Blue, and sodium or calcium salt of [4-(α-(4-diethylaminophenyl)-5-hydroxy-2,4-disulfophenylmethylidene)-2,5cyclohexadien-1-ylidene] diethylammonium hydroxide of formula (II) (also known as BluPatent) it is possible to prepare colored and stable aqueous or hydro-alcoholic solutions, which maintain substantially unaltered the bactericidal power of chlorhexidine, and which can be conveniently used for the disinfection and identification of the patient local area, associated to the pre-surgical procedures.

The composition of the invention is particularly suitable, for example, for the disinfection of the point of insertion of venous or arterial catheters, for antisepsis of skin areas in the injection therapy, and in the preparation of the skin prior to invasive procedures, such as biopsies, downspouts, diagnostic punctures and surgeries.

### SUMMARY OF THE INVENTION

In a first aspect, the invention relates to an antimicrobial coloring composition comprising at least:

chlorhexidine or a pharmaceutically acceptable salt thereof, and a dye selected from: 3,7-bis(dimethylamino)-phenothiazin-5-ium chloride of formula (I), and a sodium or calcium salt of [4-(α-(4-diethylaminophenyl)-5-hydroxy-2,4-disulfophenylmethylidene)-2,5-cyclohexadien-1-ylidene] diethylammonium hydroxide of formula (II):

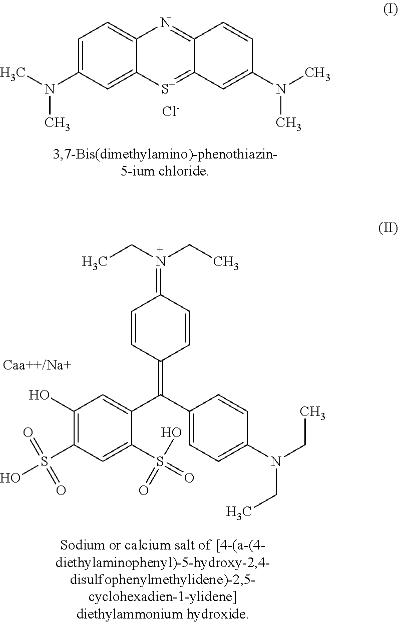

In a further aspect the invention relates to the above composition, in the form of a gel or aqueous or hydro alcoholic solution, these latter obtained respectively by mixing said composition with water or with a solvent system comprising water and at least one lower C₁-C₄ alkyl alcohol.

In a further aspect, the invention relates to said composition for use as a medicament, in particular as antimicrobial coloring agent in the pre-surgical procedures.

Constitutes an additional aspect of the invention a method for pre-surgical disinfection and identification of an area to be subjected to surgery, said method comprising the pre-surgical skin application of a hydro-alcoholic solution of the invention, in correspondence of the area of the human or animal body to operate. This application can take place for example by contacting the affected skin area with the solution of the invention.

### DETAILED DESCRIPTION

The term “% by weight” (% w/w) indicates the weight of the component with respect to the total weight.

The term “aqueous solution” includes in its meaning those solutions obtained by mixing the composition of the invention with water, and having an appearance substantially free from precipitates or solid residues.

Similarly, the term “hydro-alcoholic solution” includes in its meaning those solutions obtained by mixing the composition of the invention in a hydro-alcoholic solvent system comprising water and at least one lower C₁-C₄ alkyl alcohol, as described herein below in detail.

These solutions (aqueous or hydro-alcoholic) are generally substantially free of solid residues, typical for example of a suspension, and may be saturated, i.e. having reached the maximum degree of solubility of the components, or having various concentrations.

For “hydro-alcoholic solvent system” is intended to indicate a liquid mixture comprising water and at least one lower C₁-C₄ alkyl alcohol, capable of dissolving the present composition, without leaving substantial precipitates or solid residues.

The term “lower C₁-C₄ alkyl alcohol” means a linear or branched alkyl alcohol compound having 1 to 4 carbon atoms, such as for example: ethanol, propanol, isopropanol and the like.

As above indicated, the present composition allows to obtain a medicament stable over the time, and widely usable in the medical field as coloring and antimicrobial agent.

In particular, the composition of the invention may be useful in the pre-surgical procedures for the detection and disinfection, preferably at skin level, of the body areas to be subjected to subsequent surgical treatment.

In one embodiment, the composition of the invention may be admixed with water or, preferably, with a hydro-alcoholic solvent system, giving rise in this way, to aqueous or hydro-alcoholic solutions respectively.

The present composition, in fact, is characterized by the fact of being usable for the preparation of solutions even having a high alcoholic percentage (in the order of 60-70% w/w) and a high chlorhexidine concentrations (which can be up to 4% p/p), keeping substantially unchanged over the time the disinfecting action and the color of the thus obtained solutions.

In one form of the invention, the chlorhexidine is present as a salt thereof, even more preferably in amounts between about 0.1% w/w and 4% w/w, preferably between about 1% w/w and 4% w/w, compared to the total weight.

Preferably, said salt is selected from: gluconate, digluconate, acetate and hydrochloride, being the digluconate (CAS No: 18472-51-0) particularly preferred.

The dye used in the present invention is preferably the 3,7-bis(dimethylamino)-phenothiazin-5-ium chloride of formula (I) (CAS No. 61-73-4), also known as Methylene Blue.

Equally preferred is the dye of formula (II), in the form of sodium salt (CAS 20262-76-4) or, preferably, as calcium salt (CAS 3536-49-0).

The present composition comprises the dye of formula (I) or (II) in an amount preferably between about 0.01% and 0.1% w/w, being an amount between about 0.04% w/w and 0.08% w/w particularly preferred.

The present antiseptic coloring composition is preferably in the form of hydro-alcoholic solution, being hydro-alcoholic solutions comprising an amount of lower C₁-C₄ alkyl alcohol between about 60% w/w and 70% w/w particularly preferred. In one embodiment, the hydro-alcoholic solution comprises water and at least one lower C₁-C₄ alkyl alcohol selected from: ethyl alcohol and isopropyl alcohol.

Particularly preferred is a hydro-alcoholic solution comprising about 70% w/w of ethyl or isopropyl alcohol, and about 4% w/w of chlorhexidine digluconate. Surprisingly, applicants have noted that even in the presence of these high concentrations of active ingredients the final solutions are stable over the time and also in line with the current regulations provided by law (e.g. protocols CIPAC 46 and CIPAC 39.3), as shown in the below experimental part. In this regard, it should be noted the fact that high stability can also be obtained by formulating the present composition in a gel form, as in Example 5 of the present experimental part. Appropriate gel may be obtained by known techniques in the field, for example by mixing with synthetic polymers, such as polyvinyl pyrrolidones (for example Povidone K30 or Kovidone) and the like, and with hydroxypropyl cellulose or ethyl cellulose.

Hydro-alcoholic solutions can be prepared by dissolving the composition of the invention in a suitable solvent system comprising water and at least one lower C₁-C₄ alkyl alcohol, as indicated above. Preferably, the solvent system of the invention comprises ionized, distilled, by injection or, even more preferably, osmotic water. Due to the high solubility of the components, the hydro-alcoholic solution of the invention can be obtained in a short frame of time by the addition to the chosen solvent system of the individual components (salt of chlorhexidine and dye (I) or (II)), for example in the form of a homogeneous mixture, or singly. Hydro-alcoholic solutions including alcohol, preferably isopropyl alcohol or ethyl alcohol, and water in a ratio ranging between 60/40 and 70/30 are preferred ones.

In a similar way it is possible to prepare aqueous solutions comprising the composition of the invention, preferably using deionized, distilled, or, even more preferably, osmotic water.

In any case, the thus obtained solutions present a high degree of stability, being able to retain substantially unaltered the antimicrobial (antiseptic and disinfectant) activity of the chlorhexidine, being also able to perform the coloring function on the skin, due to the presence of the dye of formula (I) or (II).

Advantageously, therefore, the present invention allows the preparation and the storage for several months (or even years) of said solutions, avoiding in this way the preparation immediately prior to the surgical intervention, as it happens in the prior art in case of similar disinfecting and coloring compositions.

In a further embodiment, the present composition, for example in the form of hydro-alcoholic solution, can also include additional active ingredients having antimicrobial activity, preferably the didecyldimethylammonium chloride and/or benzalkonium chloride or polyhexamethylen biguanide.

In any case, either if the composition of the invention only contain the active principle chlorhexidine, or in the case where the chlorhexidine is in admixture with another antimicrobial agent, it is possible to obtain final solutions which are stable in time, and that, advantageously, allow an effective antibacterial and/or antiseptic action, even further enhanced by the combination of two or more active ingredients. Thanks to the possibility of a cutaneous administration, the present composition not only allow to visually indicate the site of the human or animal body which has to be subjected to surgery (thanks to the presence of the dye of formula (I) or (II)), but also it makes possible to exploit a wide-range antimicrobial activity, especially useful in the case of invasive transcutaneous interventions such as injections, biopsies or the like.

Therefore, in an additional aspect, the invention relates to the use of the present composition, preferably in the form of aqueous or hydro-alcoholic solutions, for the preparation of a medicament with coloring and antimicrobial action. In one embodiment, the use is particularly suitable for cutaneous administration.

This administration can be done either on an animal or on human body, usually by rubbing, dripping or similar procedures.

The present invention will now be described with the following experimental part without however limiting the scope.

### EXPERIMENTAL PART

The following hydro-alcoholic solutions and gels were prepared and tested with regards to their stability.

### Example 1

### Preparation of a Hydro-Alcoholic Solution Comprising: Chlorhexidine 2%, Blue Dye of Formula (I) and Ethanol

Composition: Chlorhexidine digluconate 2% w/w, Ethyl Alcohol (95%) 70% w/w, 3,7-bis(Dimethylamino)-phenothiazin-5-ium chloride (Methylene Blue) 0.08% w/w.

General procedure: 100 grams of Chlorhexidine digluconate (20%) were added with stirring to 200 grams of distilled water. Then 700 g of Ethyl Alcohol (95%) and 0.8 g of Methylene Blue were added thereto. Mixing for 10 min and packaging.

### Example 2

### Preparation of a Hydro-Alcoholic Solution Comprising: Chlorhexidine 2%, Blue Dye of Formula (I) and Iso-Propanol

Composition: Chlorhexidine digluconate 2% Isopropyl Alcohol (99%) 70% w/w, 3,7-bis(Dimethylamino)-phenothiazin-5-ium chloride (Methylene Blue) 0.08% w/w.

General procedure: 100 grams of Chlorhexidine digluconate (20%) were added with stirring to 200 grams of distilled water. Then 700 g of iso-propylic alcohol (95%) and 0.8 g of Methylene Blue were added thereto. Mixing for 10 min and packaging.

### Example 3

### Preparation of a Hydroalcoholic Solution Comprising: Chlorhexidine 4%, Blue Dye of Formula (I) and Ethanol

Composition: Chlorhexidine digluconate 4%, Ethyl Alcohol (95%) 70%, 3,7-bis(Dimethylamino)-phenothiazin-5-ium chloride (Methylene Blue) 0.08% w/w.

General procedure: 200 g of Chlorhexidine digluconate (20%) were added under stirring to 100 grams of distilled water. Then 700 g of Ethyl Alcohol (95%) and 0.8 g of methylene blue were added thereto. Mixing for 10 min and packaging.

### Example 4

### Preparation of an Aqueous Solution Comprising: Chlorhexidine 4%, Blue Dye of Formula (I) and Water

Composition: Chlorhexidine digluconate 4%, 3,7-bis(Dimethylamino)-phenothiazin-5-ium chloride (Methylene Blue) 0.08% w/w, distilled water to 100.

General procedure: 200 grams of Chlorhexidine digluconate (20%) were added under stirring to 800 g of distilled water. Then 0.8 g of methylene blue were added thereto. Mixing for 10 min and packaging.

### Example 5

### Preparation of a Gel Using the Composition of the Invention COMPRISING 4% Chlorhexidine, Blue Dye of Formula (I) and Water

Composition: Chlorhexidine digluconate 4%, chloride, 3,7-bis(Dimethylamino)-phenothiazin-5-ium chloride (Methylene Blue) 0.08% w/w, distilled water to 100, Hydroxypropyl cellulose 0.8-1.2%; Kovidone K30 0.6-0.8%.

General procedure: 200 grams of Chlorhexidine digluconate (20%) were added under stirring to about 800 grams of distilled water. Then 0.8 g of Methylene blue, hydroxypropyl cellulose in the range of 0.8-1.2%, and KoVidone K30 in the range of 0.6-0.8% were added thereto. Mixing for 10 min and packaging.

### Example 6

Analogous preparations can be realized using the dye Blue patent of formula (II) in place of methylene blue, obtaining stability results comparable with those related to preparations of Examples 1-5 above.

### STABILITY TEST OF THE COMPOSITIONS OBTAINED IN THE EXAMPLES 1-6

In all the cases of the above examples 1-6, the accelerated stability was evaluated according to standard protocols that encompasses the maintenance of the solutions or gels of the Examples 1-6 at a temperature of about 54±2° C., for a period of time equal to 14 days (study protocol CIPAC 46), and the maintenance of the same at 0±2° C. for a period of time of 7 days (study Protocol CIPAC 39.3).

Sample analysis includes both the initial and final control of concentrations of the various components, and the observation of the presence of stratification/precipitates.

All the samples of the Examples 1-6 gave positive results to the protocols of accelerated stability.

Worth to note that the obtained results of accelerated testing protocols provided by the CIPAC 46 and CIPAC 39.3, are indicative of a stability of the compositions corresponding to a period of time of 2 years.

1. An antiseptic coloring composition, in the form of a hydro-alcoholic solution comprising at least one lower C₁-C₄ alkyl alcohol in an amount between 60% w/w and 70% w/w, chlorhexidine or a pharmaceutically acceptable salt thereof, and the dye 3,7-bis(dimethylamino)-phenothiazin-5-ium chloride of formula (I), in amounts between 0.04 w/w and 0.08% w/w.

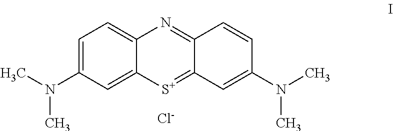

2. The composition according to claim 1, comprising a chlorhexidine salt selected from the group consisting of: gluconate, digluconate, acetate and hydrochloride.

3. The composition according to claim 2, wherein said chlorhexidine salt is the digluconate.

4. The composition according to claim 1, comprising chlorhexidine or a pharmaceutically acceptable salt thereof, in amounts between 0.1% w/w and 4% w/w.

5. The composition according to claim 4, comprising chlorhexidine or a pharmaceutically acceptable salt thereof in amounts between 1% w/w and 4% w/w.

6. The composition of claims 5, wherein said at least one lower C₁-C₄ alkyl alcohol, is selected from the group consisting of: ethanol and isopropanol.

7. The composition according to claim 3, comprising a lower C₁-C₄ alkyl alcohol 70% w/w, and chlorhexidine digluconate 4% w/w.

8. The composition according to claim 1, further comprising at least one compound with antimicrobial activity, selected from the group consisting of: didecyldimethylammonium chloride, benzalkonium chloride, polyhexamethylene biguanide and mixtures thereof.

9. The composition according to claim 1, comprising the lower C₁-C₄ alkyl alcohol and water in a ratio ranging between 60/40 and 70/30.

10. (canceled)

11. (canceled)

12. (canceled)

13. (canceled)

14. A method for pre-surgical disinfection and identification of an area to be subjected to surgery, said method comprising the pre-surgical skin application of the hydro-alcoholic solution of claim 1, to the area of the human or animal body to be treated.

15. A method for pre-surgical disinfection and identification of an area to be subjected to surgery, said method comprising the pre-surgical skin application of the hydro-alcoholic solution of claim 6, to the area of the human or animal body to be treated.
